(19) United States
(12) Patent Application Publication
(10) Pub. No.: US 2021/0162209 A1
(43) Pub. Date: Jun. 3, 2021

(54) Lightweight Iontophoresis Device

(71) Applicant: UBDEVICE CORP., Taoyuan City (TW)

(72) Inventor: Wei-Chieh Chen, Taoyuan City (TW)

(21) Appl. No.: 16/699,715

(22) Filed: Dec. 1, 2019

(51) Int. Cl. A61N 1/32; A45D 44/00; A61N 1/04

(52) U.S. Cl. CPC A61N 1/325; A45D 44/002; A45D 2200/202; A61N 1/0476; A61N 1/0496

(57) ABSTRACT

An iontophoresis device may include a flexible support layer, an electrode layer, having at least two electrically isolated electrodes, fixed to the support layer. A return electrode is separated from the electrode layer and the flexible support layer. A control module is detachably connected to the electrode layer and to the return electrode. The control module is configured to provide a variable current source or a variable voltage source only to a selected proper subset of the at least two electrically isolated electrodes while not providing the variable current source to other electrodes of the at least two electrically isolated electrodes.

### BACKGROUND OF THE INVENTION

### 1. Field of the Invention

This disclosure relates to an iontophoresis device, and more specifically to a lightweight iontophoresis device utilizing individually activated electrodes.

### 2. Description of the Prior Art

Various iontophoresis devices have been proposed to improve application of cosmetics or medical treatments with the use of electricity. For example, China Patent Application Number 200951261Y discloses a facial mask having a conductive net and an electrode patch. When a current of positive and negative ions is released, the positive and negative ions produced by the current are distributed on the conductive net layer of the ion facial mask and the effective components on the cosmetic facial mask are permeated and introduced into the skin by alternating running ions.

Another example is found in PCT Patent Application WO2016016015A1. Here, a support for a facial mask includes both an electrode and a counter separated from one another by an electrically insulating zone.

However, prior art iontophoresis devices are not able to be properly adjusted depending upon the specific skin areas to which they are being applied.

### SUMMARY OF THE INVENTION

An iontophoresis device may comprise a flexible support layer, an electrode layer fixed to the support layer, the electrode layer comprising at least two electrically isolated electrodes, a return electrode separated from the electrode layer and the flexible support layer, and a control module detachably connected to the electrode layer and to the return electrode. The control module is configured to provide a variable current source or a variable voltage source only to a selected proper subset of the at least two electrically isolated electrodes while not providing the variable current source to other electrodes of the at least two electrically isolated electrodes. Each of the at least two electrically isolated electrodes terminates in at least one exposed electrically conductive application point. The flexible support layer may comprise a non-conductive material such as synthetic resin and/or a polymer and the electrode layer may be fixed to the support layer by glue or lamination.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates a lightweight iontophoresis device, shown as a facial mask, according to an embodiment of the invention.

FIG. 2 illustrates an example electrode layer in the iontophoresis device of FIG. 1.

FIG. 3 illustrates an embodiment of a control circuit that utilizes a variable current source.

FIG. 4 illustrates another embodiment of a control circuit that utilizes a variable current source.

FIG. 5 illustrates an embodiment of a control circuit that utilizes a variable voltage source.

FIG. 6 illustrates an embodiment of a control circuit that utilizes a form of multiplexor for electrode selection.

### DETAILED DESCRIPTION

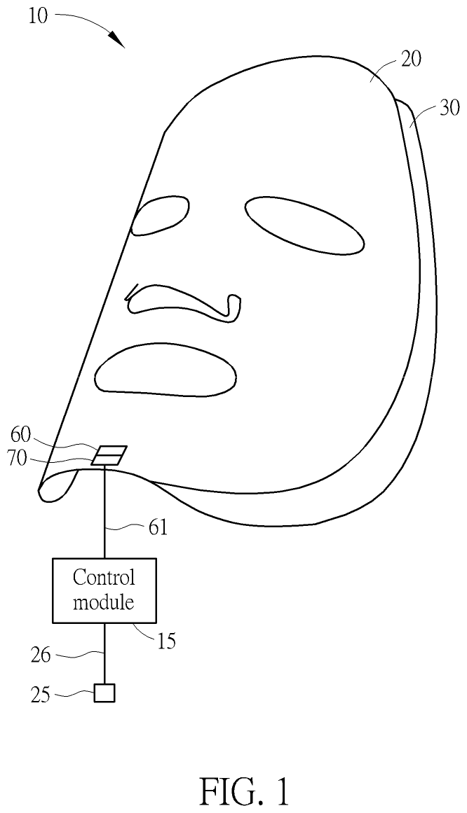

A lightweight iontophoresis device 10 for direct application of medical or cosmetic treatment to the skin is disclosed in FIG. 1. The iontophoresis device 10 is shown as a facial mask although it may be any shape and used on almost any part of the body requiring treatment. The iontophoresis device 10 may comprise a support layer 20, an electrode layer 30, a control module 15, and return electrode 25, along with connecting cables 26, 61 and interface 60. The support layer 20 may comprise a flexible, non-conductive material such as synthetic resin or a polymer, but is not limited to these examples. The electrode layer 30 may be integrated into the support layer 20 or attached to the support layer 20, perhaps by glue or lamination technics. The electrode layer 30 may be some conductive patterns directly made on the support layer 20 by printing, coating, sputtering or other patterning techniques.

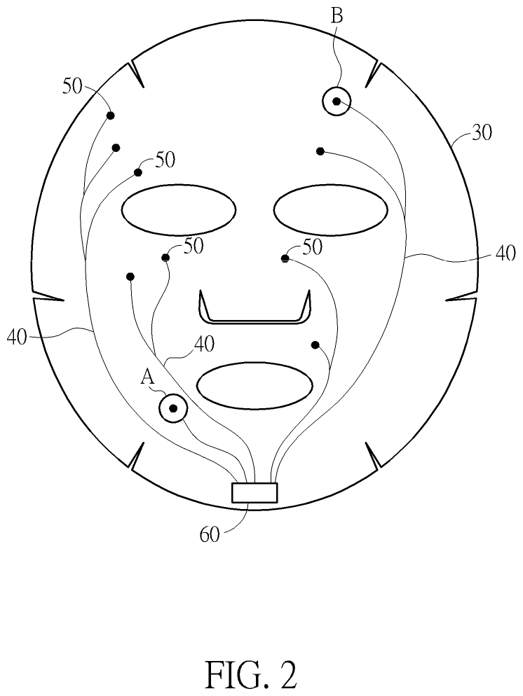

An example electrode layer 30 is better shown in FIG. 2. The electrode layer 30 may comprise a plurality of electrically isolated electrodes 40. Each of the electrodes 40 may comprise one or more branches having conductive application points 50. Each of the electrodes 40 may comprise the same number of application points 50 or the number of branches and/or application points 50 may vary from electrode 40 to electrode 40. The number and location of each application point 50 on each electrode 40 may be subject to design considerations but in some embodiments may depend upon the underlying skin, muscle group, acupuncture points, and/or other designated areas to which the application points 50 are applied. In some embodiments, the application points 50 can be omitted from one or some or all the electrodes 40. The electrodes 40 can be used to directly provide iontophoresis treatment to more area of the face or the body according to the electrode pattern design.

Each of the electrodes 40 is individually connected, via connector 60 and cable 61, to a control module 15. The control module 15 is further electrically connected, via cable 26, to a return electrode 25 attached to part of the body other than where the iontophoresis device 10 is placed. One purpose of the return electrode 25 is to complete an electrical circuit from the control module 15, to the designated application points 50 through the electrodes 40, then through the body to the return electrode 25, and back to the control module 15.

The control module 15 is configured to connect the electrodes 40 with a voltage or current source. Each electrode 40 may be individually connected or not connected to the electrical source to provide medical or cosmetic iontophoresis treatment only to selected portions of the face or body. For example, in FIG. 2, only the right-most electrode 40 (and the attached application points 50) may receive electricity when only that part of the forehead requires treatment. Any number of electrodes 40 may receive electricity at any particular time. However, research shows that even if all electrodes are to be used, turning on (receive electricity) only one or a subset of the electrodes 40 at a time is more effective at least because it allows more flexibility in the current or voltage used.

Regardless of where on the body the iontophoresis device 10 is placed, the total impedance of a circuit involving a single electrode 40 will be different from the impedance of a circuit involving a different single electrode 40. For example, due to many factors including skin moisture levels and distances between the application point 50 and the return electrode 25, the impedance of a circuit going through application point 50 marked in FIG. 2 as “A” is different than the impedance of a circuit going through application point 50 marked as “B”.

If all electrodes 40 are turned on at the same time, the different impedances make it difficult to accurately control the electrical flow and some skin areas will receive more electricity than others resulting in an uneven treatment. Because the electrodes 40 in the iontophoresis device 10 are individually activated and controlled, the differences in impedances between electrodes 40 can be compensated, resulting in a more even treatment. Additionally, sometimes only a specific skin area requires treatment or a specific skin area requires treatment for a different length of time, which can be easily achieved by only turning on electrodes 40 affecting that specific skin area. In some embodiments, different levels of electricity can be applied to different areas as needed.

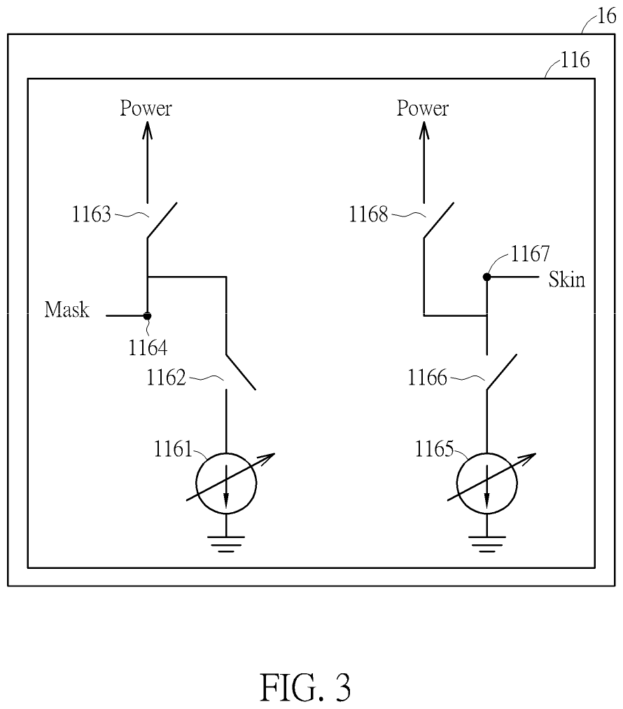

FIGS. 3-6 illustrate examples of a control unit 16 used to selectively activate the electrodes 40.

FIG. 3 illustrates a first embodiment of a circuit 116 that utilizes a variable current source 1161 and/or 1165. When the circuit 116 is connected to the iontophoresis device 10, the variable current source 1161 is serially coupled between ground and a first terminal of a first switch 1162. The second terminal of the first switch 1162 is coupled to a node 1164, which connects to one or more electrode 40 via cable 61 and connectors 70, 60. The second terminal of the first switch 1162 also connects to a first terminal of a second switch 1163. The second terminal of the second switch 1163 couples to power.

The circuit 116 that can be connected to the return electrode 25 via cable 26 may comprise a second variable current source 1165 serially coupled between ground and a first terminal of a third switch 1166. The second terminal of the third switch 1166 is connected to a node 1167, which connects to cable 26. The second terminal of the third switch 1166 also connects to a first terminal of a fourth switch 1168. The second terminal of the fourth switch 1168 couples to power.

In some embodiments, there will be at least one circuit 116 in the control unit 16 for each of the electrically isolated electrodes 40 so that each electrode 40 can be individually activated.

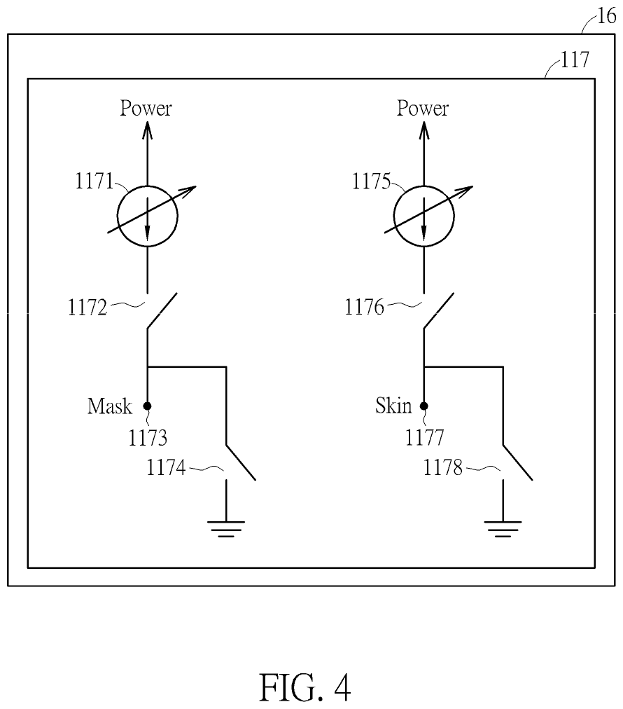

FIG. 4 illustrates another embodiment of a circuit 117 that utilizes a third variable current source 1173 and/or 1175. The variable current source 1171 is serially coupled between power and a first terminal of a fifth switch 1172. The second terminal of the fifth switch 1172 is coupled to a node 1173, which again connects to one or more electrode 40 via cable 61 and connectors 70, 60. The second terminal of the fifth switch 1172 also connects to a first terminal of a sixth switch 1174. The second terminal of the sixth switch 1174 couples to ground as shown.

The circuit 117 that can be connected to the return electrode 25 via cable 26 may comprise a fourth variable current source 1175 serially coupled between power and a first terminal of a seventh switch 1176. The second terminal of the seventh switch 1176 is connected to a node 1177, which connects to cable 26. The second terminal of the seventh switch 1176 also connects to a first terminal of an eighth switch 1178. The second terminal of the eighth switch 1178 couples to ground.

In some embodiments of FIG. 4, there will be at least one circuit 117 in the control unit 16 for each of the electrically isolated electrodes 40 so that each electrode 40 can be individually activated.

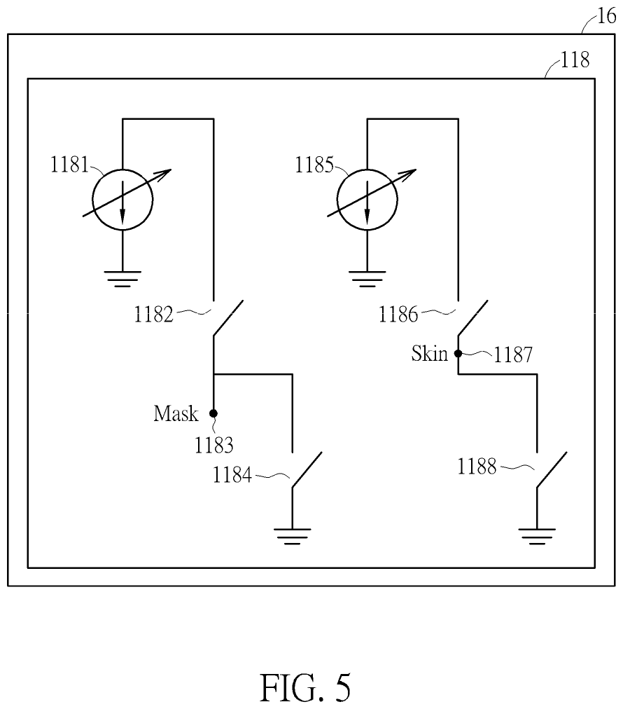

FIG. 5 illustrates an embodiment of a circuit 118 that utilizes a first variable voltage source 1181 and/or 1185. The variable voltage source 1181 is serially coupled between ground and a first terminal of a ninth switch 1182. The second terminal of the fifth switch 1182 is coupled to a node 1183, which again connects to one or more electrode 40 of the iontophoresis device 10 via cable 61 and connectors 70, 60. The second terminal of the ninth switch 1182 also connects to a first terminal of a tenth switch 1184. The second terminal of the tenth switch 1184 couples to ground as shown.

The circuit 118 that can be connected to the return electrode 25 via cable 26 may comprise a second variable voltage source 1185 serially coupled between ground and a first terminal of an eleventh switch 1186. The second terminal of the eleventh switch 1186 is connected to a node 1187, which connects to cable 26. The second terminal of the eleventh switch 1186 also connects to a first terminal of a twelfth switch 1188. The second terminal of the twelfth switch 1878 couples to ground.

In some embodiments of FIG. 5, there will be at least one circuit 118 in the control unit 16 for each of the electrically isolated electrodes 40 so that each electrode 40 can be individually activated.

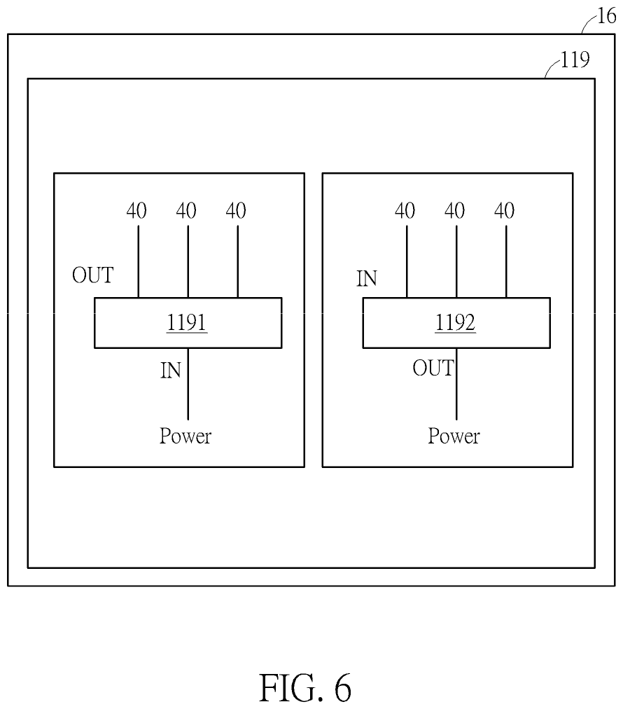

FIG. 6 illustrates an embodiment of a circuit 119 that utilizes a form of multiplexer 1192 or de-multiplexor 1191 as the switching device. De-multiplexor 1191 receives the appropriate current or voltage at the input and routes it to the appropriate electrode 40.

Circuit 119 may also or alternatively include the multiplexor 1192, which receives current or voltage from a selected electrode 40 and outputs the input to complete the circuit.

In all of the above embodiments, the control terminals of the switches and/or multiplexor device may be regulated manually or via a computer using the control module.

In summary, a lightweight iontophoresis device for direct application of medical or cosmetic treatment to the skin is proposed. The iontophoresis device may comprise an electrode layer having a plurality of individually selectable, electrically isolated conductive electrodes, each electrode comprising one or more branches having application points. A control module is configured to individually connect the electrodes with a voltage or current source to provide medical or cosmetic iontophoresis treatment only to selected portions of the face or body, providing a more effective iontophoresis treatment at least because it allows more flexibility in the current or voltage used when treating a specific area of skin. Additionally, because only selected areas of skin are treated at a particular time, a smaller current or voltage may be required resulting in a more pleasing experience for the recipient.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An iontophoresis device comprising:
a flexible support layer;
an electrode layer fixed to the support layer, the electrode layer comprising at least two electrically isolated electrodes;
a return electrode separated from the electrode layer and the flexible support layer; and
a control module removably connected to the electrode layer, to the return electrode, the control module configured to provide a variable current source only to a selected proper subset of the at least two electrically isolated electrodes while not providing the variable current source to other electrodes of the at least two electrically isolated electrodes.

2. The iontophoresis device of claim 1 wherein each of the at least two electrically isolated electrodes terminates in at least one exposed electrically conductive application point.

3. The iontophoresis device of claim 1 wherein the variable current source is serially coupled between ground and a first terminal of a first switch, a second terminal of the first switch is coupled to one of the at least two electrically isolated electrodes, the second terminal of the first switch also connects to a first terminal of a second switch, and a second terminal of the second switch is coupled to power.

4. The iontophoresis device of claim 1 wherein the variable current source is serially coupled between ground and a first terminal of a third switch, a second terminal of the third switch is coupled to the return electrode, the second terminal of the third switch also connects to a first terminal of a fourth switch, and a second terminal of the fourth switch is coupled to power.

5. The iontophoresis device of claim 1 wherein the variable current source is serially coupled between power and a first terminal of a fifth switch, a second terminal of the fifth switch is coupled to one of the at least two electrically isolated electrodes, the second terminal of the fifth switch also connects to a first terminal of a sixth switch, and a second terminal of the sixth switch is coupled to ground.

6. The iontophoresis device of claim 1 wherein the variable current source is serially coupled between power and a first terminal of a seventh switch, a second terminal of the seventh switch is connected to the return electrode, the second terminal of the seventh switch also connects to a first terminal of an eighth switch, and a second terminal of the eighth switch is coupled to ground.

7. The iontophoresis device of claim 1 wherein the variable current source is coupled to an input of a de-multiplexor, each output of the de-multiplexor electrically coupled to a different one of the at least two electrically isolated electrodes.

8. The iontophoresis device of claim 1 wherein the variable current source is coupled to an output a multiplexer, input of the multiplexor electrically connected to a different one of the at least two electrically isolated electrodes.

9. The iontophoresis device of claim 1 wherein the flexible support layer comprises a non-conductive material selected from a group consisting of synthetic resin and a polymer.

10. The iontophoresis device of claim 1 wherein the electrode layer is fixed to the support layer by glue or lamination.

11. The iontophoresis device of claim 1 wherein the electrode layer comprises conductive patterns made on the support layer by printing, coating, sputtering or other patterning techniques.

12. An iontophoresis device comprising:
a flexible support layer;
an electrode layer fixed to the support layer, the electrode layer comprising at least two electrically isolated electrodes;
a return electrode separated from the electrode layer and the flexible support layer; and
a control module removably connected to the electrode layer, to the return electrode, the control module configured to provide a variable voltage source only to a selected proper subset of the at least two electrically isolated electrodes while not providing the variable voltage source to other electrodes of the at least two electrically isolated electrodes.

13. The iontophoresis device of claim 12 wherein each of the at least two electrically isolated electrodes terminates in at least one exposed electrically conductive application point.

14. The iontophoresis device of claim 12 wherein the variable voltage source is serially coupled between ground and a first terminal of a ninth switch, a second terminal of the ninth switch is coupled to one of the at least two electrically isolated electrodes, the second terminal of the ninth switch also connects to a first terminal of a tenth switch, and a second terminal of the tenth switch is coupled to ground.

15. The iontophoresis device of claim 12 wherein the variable voltage source is serially coupled between ground and a first terminal of an eleventh switch, a second terminal of the eleventh switch is coupled to the return electrode, the second terminal of the eleventh switch also connects to a first terminal of a twelfth switch, and a second terminal of the twelfth switch is coupled to ground.

16. The iontophoresis device of claim 12 wherein the variable voltage source is coupled to an input of a de-multiplexor, each output of the de-multiplexor electrically coupled to a different one of the at least two electrically isolated electrodes.

17. The iontophoresis device of claim 12 wherein the variable voltage source is coupled to an output a multiplexer, input of the multiplexor electrically connected to a different one of the at least two electrically isolated electrodes.

18. The iontophoresis device of claim 12 wherein the flexible support layer comprises a non-conductive material selected from a group consisting of synthetic resin and a polymer.

19. The iontophoresis device of claim 12 wherein the electrode layer is fixed to the support layer by glue or lamination.

20. The iontophoresis device of claim 12 wherein the electrode layer comprises conductive patterns made on the support layer by printing, coating, sputtering or other patterning techniques.
